(19) United States
(12) Patent Application Publication
(10) Pub. No.: US 2019/0172563 A1
(43) Pub. Date: Jun. 6, 2019

(54) FRICTIONLESS PROCESSING FOR AUTOMATIC ADJUDICATION OF MEDICAL ENCOUNTERS

(71) Applicant: CERNER INNOVATION, INC., KANSAS CITY, KS (US)

(72) Inventors: James L. Poteet, III, Overland Park, KS (US); Raymond G. Delano, III, Kansas City, MO (US)

(21) Appl. No.: 15/830,336

(22) Filed: Dec. 4, 2017

(51) Int. Cl. G16H 10/60; G06Q 20/14; G06Q 20/10

(52) U.S. Cl. CPC G16H 10/60; G16H 15/00; G06Q 20/10; G06Q 20/14

(57) ABSTRACT

Systems and methods for providing automatic adjudication (auto-adjudication) of medical encounters are provided. For example, specific encounters corresponding to a billable service provided to a patient may bypass insurance verification billing, code validation, and/or claim scrubbing. The billable service may have a program bundle that can be utilized to determine if the encounter is eligible for a fast pass token. The fast pass token prevents insurance verification billing, code validation, and claim scrubbing for the encounter. Additionally, the program bundle can be utilized to determine whether the encounter is eligible to participate in auto-adjudication and electronic funds transfer, enabling real-time financial transactions for the encounter.

### CROSS-REFERENCE TO RELATED APPLICATIONS

This Application is related to commonly assigned U.S. patent applications entitled “Frictionless Processing to Bypass Insurance Verification Billing” (Attorney Docket CRNI.275976), “Frictionless Processing to Bypass Code Validation” (Attorney Docket CRNI.280541), and “Frictionless Processing to Bypass Claim Scrubbing” (Attorney Docket CRNI.280542), filed concurrently herewith on the same date.

### BACKGROUND

In healthcare today, the billing process involves a number of steps that can be quite onerous. Initially, insurance must be verified to confirm a number of details. These details may include benefits, co-payments, coinsurance, deductibles, policy status, effective date, type of plan, exclusions, mailing address, referrals, pre-authorizations, and lifetime maximum. The verification process may involve visiting a payer website or calling the insurance company directly. Sometimes, it may also be necessary to contact patients to confirm contact details and demographic information. This process must be repeated for each encounter because medical coverage can change over a short period of time.

If the insurance has been verified, reimbursement cannot occur until each billable service of an encounter has been properly coded. This process often requires dedicated professionals that abstract information from documentation (e.g., notes of a clinician, laboratory results, etc.) and assign standard codes using classification systems (e.g., Healthcare Common Procedure Coding System (HCPCS) and Current Procedural Terminology (CPT)). Failure to properly code each billable service of the encounter can result in a claim being denied or reimbursement being delayed.

To avoid such denials or delays, a claim is scrubbed to make sure it satisfied all the payer rules for payment. This requires up-to-date rules for each payer a provider or healthcare facility accepts. Typically, daily batches of claims are scrubbed using the rules to make sure any edits necessary to prevent denials are completed prior to billing the payer.

After the claim has been scrubbed, it can be submitted to the payer by either paper billing or electronic claim submission. Paper billing often takes six or more weeks for processing. However, even today's electronic claims processing has inherent delays. For instance, many insurance companies and payers utilize a clearinghouse. A clearinghouse is an intermediary company that receives the claims and forwards them to insurance companies for processing. Even when direct billing is utilized, there is no current method to process real-time financial transactions so a provider can be paid at the time of the encounter or when the service occurs prior to claim generation. Moreover, the current methods all incur additional costs for insurance verification, coding, claim scrubbing, and each intermediary company that is utilized during the process.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present disclosure relate to systems and methods for providing automatic adjudication (auto-adjudication) of medical encounters. More particularly, embodiments of the present disclosure enable specific encounters to bypass normal, standard encounter financial processing checks to ensure clean claims. For example, in various embodiments of the present disclosure, specific encounters may bypass insurance verification billing, code validation, and/or claim scrubbing. In some embodiments, the encounter may be auto-adjudicated with an electronic funds transfer (EFT) into the bank account of the provider.

To do so, an encounter may initially be created that corresponds to a billable service provided to a patient. The billable service may have a program bundle that can be utilized to determine if the encounter is eligible for a fast pass token. The fast pass token allows an insurance verification check for the encounter to be non-billable. In embodiments, upon determining the encounter is eligible for the fast pass token, a provider or health system is not billed for the insurance verification check. Additionally, a token eligible bit flag may be set for the encounter and stored with the encounter.

In some embodiments, once the billable service has been performed and, prior to evaluating a code set for accuracy, the encounter is evaluated for the existence of the fast pass token eligible bit flag. Upon determining the encounter has the fast pass token eligible bit flag set, service code validation rules are performed to determine if the service code is eligible for a fast pass token, and a fast pass token is generated and saved with the encounter. The provider or health system is not billed for code validation.

In some embodiments, during claim generation for the encounter, it is determined whether the encounter is associated with a fast pass token. Upon determining the encounter is associated with a fast pass token, claim scrubbing may be skipped for the encounter and the provider or health system is not billed for claim scrubbing.

In some embodiments, the program bundle can be utilized to determine whether the encounter with a fast pass token is eligible to participate in auto-adjudication and electronic funds transfer. If the encounter with a fast pass token is eligible for auto-adjudication and electronic funds transfer, a contract management system may be queried for health service pricing associated with the contract for the code and a health plan. Additionally, electronic funds transfer may be initiated through a banking system to debit an auto-adjudication account and credit a provider or hospital bank account for the health service pricing for the encounter. A billing record having remittance data and electronic funds transfer data may be created for an auto-adjudicated transaction fee, the billing record, and the electronic funds transfer data may be communicated to a patient financial system.

### BRIEF DESCRIPTION OF THE SEVERAL VIEWS OF THE DRAWINGS

The patent or application file contains at least one drawing executed in color. The present invention is described in detail below with reference to the attached drawing figures, wherein:

FIG. 1 is a block diagram of an exemplary operating environment suitable to implement embodiments of the present invention;

FIGS. 2A-2C depict an exemplary framework of an auto-adjudication system suitable to implement embodiments of the present invention;

FIGS. 3A-3E depict an exemplary flow diagram of a method for auto-adjudication, in accordance with an embodiment of the present invention;

FIG. 4 is a flow diagram of a method for bypassing insurance verification, in accordance with embodiments of the present invention;

FIG. 5 is a flow diagram of a method for bypassing code validation, in accordance with embodiments of the present invention;

FIG. 6 is a flow diagram of a method for bypassing claim scrubbing, in accordance with embodiments of the present invention; and

FIG. 7 is a flow diagram of a method for auto-adjudication of medical encounters, in accordance with embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” might be used herein to connote different elements of methods employed, the terms should not be interpreted as implying any particular order among or between various steps herein disclosed unless and except when the order of individual steps is explicitly stated.

Various terms are used throughout this description. Definitions of some terms are included below to provide a clearer understanding of the ideas disclosed herein:

An encounter is a record of a medical encounter that corresponds to a billable service based on an interaction between a patient and healthcare provider(s) for the purpose of providing healthcare service(s) or assessing the health status of the patient.

Insurance eligibility verification ensures healthcare benefits cover a particular procedure or healthcare service(s) provided to the patient. For example, a American National Standards Institute Accredited Standards Committee X12N Insurance 270 Eligibility and Benefits request transaction (ANSI ASCX12N 270 Eligibility & Benefits inquiry transaction) can verify patient coverage including co-pays, deductibles, inpatient days used, and other pertinent benefit data to allow payments to be collected or arrangements to be made for collecting payments prior to rendering healthcare service(s).

A Fast Pass Token, as used herein, is a unique alphanumeric that enables an encounter to be flagged as non-billable for standard HIPAA transaction processing and other financial transactions.

A Program Bundle, as used herein, includes a set of conditions that, if satisfied, enable a billable service to be automatically adjudicated. The program bundle may include real-time decision support integrated in clinical workflows that ensures adherence to evidence-based standards. The set of conditions may include a single rule set available for all participants that enables coding for billing, insurance verification, claim scrubbing, and/or self-pay collection to be bypassed. For example, if a patient is being provided a mammogram, the conditions may require the patient to be a female, over forty years of age, and not to have had a mammogram within the last twelve months. If each of these conditions are satisfied, the encounter may be automatically adjudicated and coding for billing, insurance verification, claim scrubbing, and/or self-pay collection may be bypassed.

Healthcare Common Procedure Coding System (HCPCS) and Current Procedural Terminology (CPT) codes are standard medical procedure codes that represent specific medical procedures to Medicare, Medicaid and other health insurance payers.

Claim Generation (e.g., an ANSI ASCX12N 837 claim) is a process where, after billing data has been captured, a medical claim for an insurance payer is generated and transmitted electronically.

A Contract Management System helps healthcare providers manage insurance payer contracts. The contract management system contains reimbursement rates for medical procedures administered by the provider.

Remittance Advice (e.g., ANSI ASCX12N 835 Remittance Advice) is a document supplied by the insurance payer that provides notice and explanation of reasons for payment, adjustment, denial, and/or uncovered charges of a medical claim.

Analytics, as used herein, describes the healthcare analysis activities that undertaken as a result of data collected from several areas within healthcare; claims and cost data, clinical data (collected from electronic health records (EHRs)), and patient behavior and sentiment data.

A Narrow Network represents providers in a plan's network. For example, health plans negotiate the price of medical services with certain doctors, hospitals, labs, pharmacies, and other providers so the plan, and a patient represented by the plan, pays a lower cost. If the patient visits providers who are not in network, the patient may have to pay more. Today, many insurers offer plans with “narrow” networks. These plans have a lower monthly premium, but as a trade-off, have a limited choice of providers. Many plans sold in the health insurance marketplace have narrow networks, but some employers offer them, too. If a patient has one of these plans, it is important to know which providers are in network to avoid high out-of-pocket costs.

An Integrated Delivery Network (IDN) is a network of facilities and providers that work together to offer a continuum of care to a specific geographic area or market.

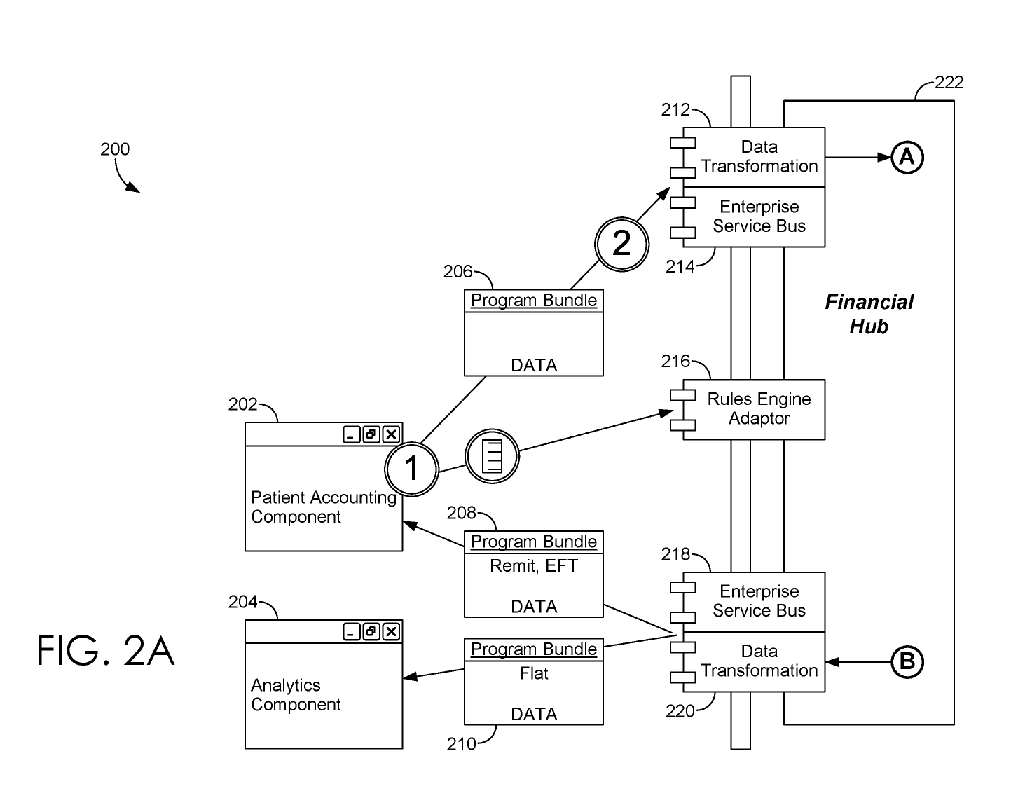

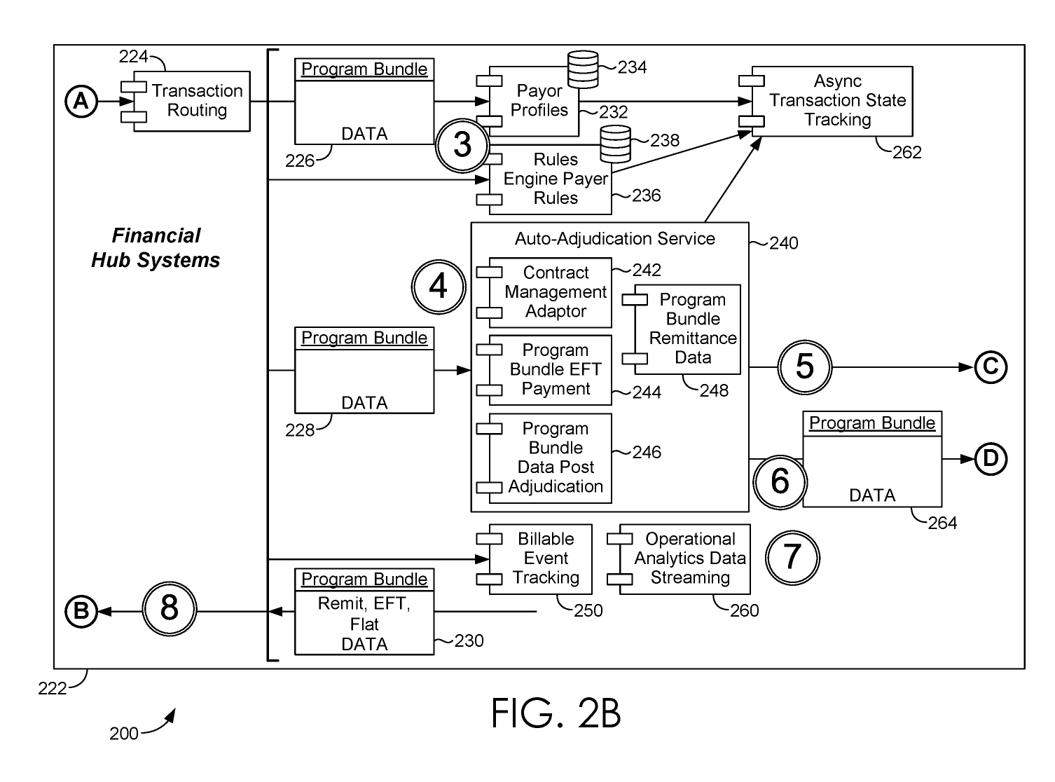

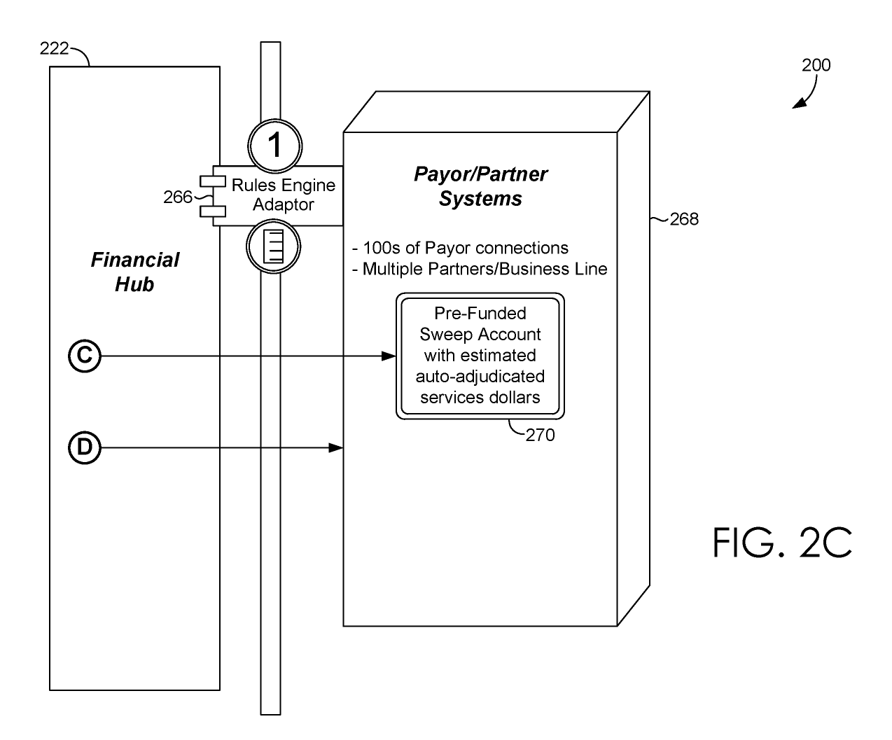

Clinically Driven Revenue Cycle Management (e.g., patient accounting component 202 of FIG. 2A) is a revenue cycle management (RCM) solution that integrates billing intelligence throughout the patient care process to speed collections, optimize revenue, eliminate financial uncertainty, and position an organization for the future.

Fiduciary process, as used herein, is the process to deposit only enough money into a bank account to fulfill one day's worth of auto-adjudication payments. The formula for funding would consist of a cost estimate of eligible services performed on a daily basis multiplied by the current contracted amount for administering each instance of an eligible service.

As noted in the background, in healthcare today, the billing process involves a number of steps that can be quite onerous. Initially, insurance must be verified to confirm a number of details. These details may include benefits, co-payments, coinsurance, deductibles, policy status, effective date, type of plan, exclusions, mailing address, referrals, pre-authorizations, and lifetime maximum. The verification process may involve visiting a payer website or calling the insurance company directly. Sometimes, it may also be necessary to contact patients to confirm contact details and demographic information. This process must be repeated for each encounter because medical coverage can change over a short period of time.

If the insurance has been verified, reimbursement cannot occur until each billable service of an encounter has been properly coded. This process often requires dedicated professionals that abstract information from documentation (e.g., notes of a clinician, laboratory results, etc.) and assign standard codes using classification systems (e.g., Healthcare Common Procedure Coding System (HCPCS) and Current Procedural Terminology (CPT)). Failure to properly code each billable service of the encounter can result in a claim being denied or reimbursement being delayed.

To avoid such denials or delays, a claim is scrubbed to make sure it satisfied all the payer rules for payment. This requires up-to-date rules for each payer a provider or healthcare facility accepts. Typically, daily batches of claims are scrubbed using the rules to make sure any edits necessary to prevent denials are completed prior to billing the payer.

After the claim has been scrubbed, it can be submitted to the payer by either paper billing or electronic claim submission. Paper billing often takes six or more weeks for processing. However, even today's electronic claims processing has inherent delays. For instance, many insurance companies utilize a clearinghouse. A clearinghouse is an intermediary company that receives the claims and forwards them to insurance companies for processing. Even when direct billing is utilized, there is no current method to process real-time financial transactions so a provider can be paid at the time of the encounter. Moreover, the current methods all incur additional costs for insurance verification, coding, claim scrubbing, and each intermediary company that is utilized during the process.

Embodiments of the present disclosure relate to systems and methods for providing auto-adjudication of medical encounters. More particularly, embodiments of the present disclosure enable specific encounters to bypass normal, standard encounter financial processing checks to ensure clean claims. For example, in various embodiments of the present disclosure, specific encounters may bypass insurance verification billing, code validation, and/or claim scrubbing. In some embodiments, the encounter may be automatically adjudicated with an EFT into the bank account of the provider. Experiments indicate at least seventy-five cents may be saved in various intermediary fees per claim by utilizing the benefits of the present disclosure.

To do so, an encounter may initially be created that corresponds to a billable service provided to a patient. The billable service may be a part of a program bundle that can be utilized to determine if the encounter is eligible for a fast pass token. In embodiments, upon determining the encounter is eligible for the fast pass token, a provider or health system is not billed for the insurance verification check. Additionally, a token eligible bit flag may be set for the encounter and stored with the encounter.

In some embodiments, once the billable service has been performed and, prior to evaluating a code set for accuracy, the encounter is evaluated for the existence of the fast pass token eligible bit flag. Upon determining the encounter has the fast pass token eligible bit flag set, service code validation rules are evaluated for fast pass token eligibility and if service code is eligible a fast pass token is generated for the encounter and the provider or health system is not billed for code validation.

In some embodiments, during claim generation for the encounter, it is determined whether the encounter is associated with a fast pass token. Upon determining the encounter is associated with a fast pass token, claim scrubbing may be bypassed for the encounter and the provider or health system is not billed for claim scrubbing.

In some embodiments, the program bundle can be utilized to determine whether the encounter is eligible to participate in auto-adjudication and electronic funds transfer. If the encounter is eligible for auto-adjudication and electronic funds transfer, a contract management system may be queried for health service pricing associated with the contract for the code and a health plan. Additionally, electronic funds transfer may be initiated through a banking system to debit an auto-adjudication account and credit a provider or hospital bank account for the health service pricing for the encounter. A billing record having remittance data and electronic funds transfer data may be created for an auto-adjudicated transaction fee, the billing record, and the electronic funds transfer data may be communicated to a patient financial system.

Accordingly, one embodiment of the present disclosure is directed to a system for utilizing frictionless processing to bypass insurance verification billing. The system includes a processor; and a computer storage medium storing computer-usable instructions that, when used by the processor, cause the processor to: create an encounter corresponding to a billable service provided to a patient, the billable service having a program bundle; and utilize the program bundle to determine if the encounter is eligible for a fast pass token, the fast pass token enables an insurance verification check for the encounter to be non-billable.

In another embodiment, the present disclosure directed to a computerized method for utilizing frictionless processing to bypass insurance verification billing. The method includes creating an encounter corresponding to a billable service provided to a patient. The billable service includes a program bundle. The method also includes utilizing the program bundle to determine if the encounter is eligible for a fast pass token, the fast pass token enabling an insurance verification check for the encounter to be non-billable. The method further includes, upon determining the encounter is eligible for the fast pass token, not billing a provider or health system for the insurance verification check.

In yet another embodiment, the present disclosure is directed to one or more computer storage media having computer-executable instructions embodied thereon that, when executed by a computer, causes the computer to perform operations to utilize frictionless processing to bypass insurance verification billing. The operations include creating an encounter corresponding to a billable service provided to a patient. The billable service includes a program bundle. The operations also include utilizing the program bundle to determine if the encounter is eligible for a fast pass token, the fast pass token enabling an insurance verification check for the encounter to be non-billable. The operations further include, upon determining the encounter is eligible for the fast pass token, not billing a provider or health system for the insurance verification check.

Another embodiment of the present disclosure is directed to a system for utilizing frictionless processing to bypass code validation. The system includes a processor; and a computer storage medium storing computer-usable instructions that, when used by the processor, cause the processor to: prior to evaluating a service code set for the billable service for accuracy, evaluate the encounter for a presence of a fast pass token eligible bit flag; and upon determining the encounter has the fast pass token eligible bit flag set and the specific service code is eligible for a fast pass token: assign a fast pass token for the encounter; store the fast pass token for the encounter in an electronic medical record (EMR); and not bill a provider or health system for code validation.

In another embodiment, the present disclosure directed to a computerized method for utilizing frictionless processing to bypass code validation. The method includes receiving an indication a billable service for an encounter has been performed. The method also includes, prior to evaluating a service code set for a billable service corresponding to an encounter for accuracy, evaluating the encounter for a presence of a fast pass token eligible bit flag. The method further includes, upon determining the encounter has the fast pass token eligible bit flag set: determining if the service code set is included in a list of services eligible for auto-adjudication, assigning a fast pass token for the encounter; storing the fast pass token for the encounter in an electronic medical record (EMR); preventing code validation for the encounter; and not billing a provider or health system for code validation.

In yet another embodiment, the present disclosure is directed to one or more computer storage media having computer-executable instructions embodied thereon that, when executed by a computer, causes the computer to perform operations to utilize frictionless processing to bypass code validation. The operations include receiving an indication a billable service for an encounter has been performed. The operations also include, prior to evaluating a service code set for a billable service corresponding to an encounter for accuracy, evaluating the encounter for a presence of a fast pass token eligible bit flag. The operations further include, upon determining the encounter has the fast pass token eligible bit flag set: determining if the service code set is included in a list of services eligible for auto-adjudication, assigning a fast pass token for the encounter; storing the fast pass token for the encounter in an electronic medical record (EMR); preventing code validation for the encounter; and not billing a provider or health system for code validation.

Another embodiment of the present disclosure is directed to a system for utilizing frictionless processing to bypass claim scrubbing. The system includes a processor; and a computer storage medium storing computer-usable instructions that, when used by the processor, cause the processor to: determine whether the encounter is associated with a fast pass token; upon determining the encounter is associated with a fast pass token: bypass claim scrubbing for the encounter; and not bill the provider or health system for claim scrubbing.

In another embodiment, the present disclosure directed to a computerized method for utilizing frictionless processing to bypass claim scrubbing. The method includes determining whether an encounter corresponding to a billable service is associated with a fast pass token, the fast pass token preventing billing a health system for claim scrubbing. Upon determining the encounter is associated with a fast pass token: claim scrubbing is bypassed for the encounter; and the provider or health system is not billed for claim scrubbing.

In yet another embodiment, the present disclosure is directed to one or more computer storage media having computer-executable instructions embodied thereon that, when executed by a computer, causes the computer to perform operations to utilize frictionless processing to bypass claim scrubbing. The operations include determining whether an encounter corresponding to a billable service is associated with a fast pass token. The fast pass token prevents billing a provider or health system for claim scrubbing. The operations further include, upon determining the encounter is associated with a fast pass token: bypassing claim scrubbing for the encounter; and not billing the provider or health system for claim scrubbing.

Another embodiment of the present disclosure is directed to a system for utilizing frictionless processing auto-adjudication of medical encounters. The system includes a processor; and a computer storage medium storing computer-usable instructions that, when used by the processor, cause the processor to: communicate claim data to a financial hub, the claim data corresponding to a billable service of an encounter, the encounter having a program bundle; utilize the program bundle to verify codes for the billable service for participation in auto-adjudication and electronic funds transfer; upon determining the billable service of the encounter is eligible for auto-adjudication and electronic funds transfer: query a contract management system for a health service price associated with a contract for the codes and a health plan of a patient; and initiate electronic funds transfer to debit an auto-adjudication account of a payer and credit a provider or hospital bank account for the health service price.

In another embodiment, the present disclosure directed to a computerized method for utilizing frictionless processing auto-adjudication of medical encounters. The method includes communicating claim data to a financial hub. The claim data corresponds to a billable service of an encounter having a program bundle. The method also includes utilizing the program bundle to verify codes for the billable service for participation in auto-adjudication and electronic funds transfer. The method further includes, upon determining the billable service of the encounter is eligible for auto-adjudication and electronic funds transfer: querying a contract management system for a health service price associated with a contract for the codes and a health plan of a patient; and initiating electronic funds transfer to debit an auto-adjudication account of a payer and credit a provider or hospital bank account for the health service price.

In yet another embodiment, the present disclosure is directed to one or more computer storage media having computer-executable instructions embodied thereon that, when executed by a computer, causes the computer to perform operations utilizing frictionless processing auto-adjudication of medical encounters. The operations include communicating claim data to a financial hub. The claim data corresponds to a billable service of an encounter having a program bundle. The operations also include utilizing the program bundle to verify codes for the billable service for participation in auto-adjudication and electronic funds transfer. The operations further include, upon determining the billable service of the encounter is eligible for auto-adjudication and electronic funds transfer: querying a contract management system for a health service price associated with a contract for the codes and a health plan of a patient; and initiating electronic funds transfer to debit an auto-adjudication account of a payer and credit a provider or hospital bank account for the health service price.

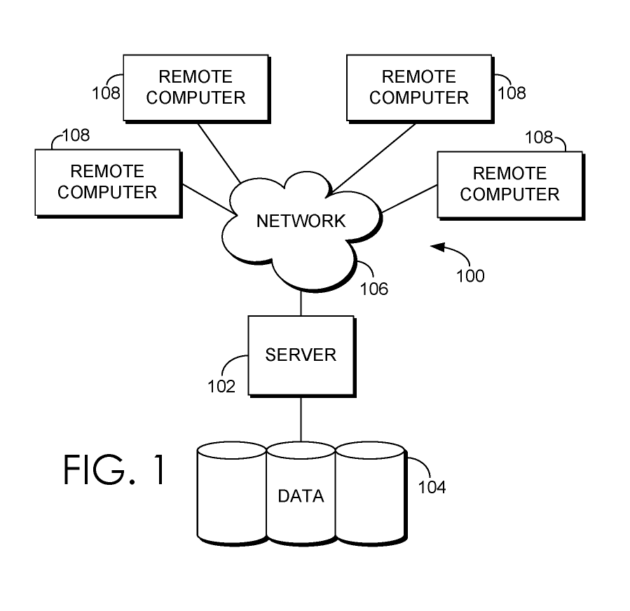

Having briefly described embodiments of the present invention, an exemplary operating environment suitable for use in implementing embodiments of the present invention is described below. FIG. 1 provides an aspect of an example operating environment with which embodiments of the present invention may be implemented. The aspect of an operating environment is illustrated and designated generally as reference numeral 100.

Example operating environment 100 comprises a general purpose computing device in the form of a control server 102. Exemplary components of the control server 102 comprise a processing unit, internal system memory, and a suitable system bus for coupling various system components, including database cluster 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Control server 102 typically includes therein, or has access to, a variety of computer-readable media, for instance, database cluster 104. Computer-readable media can be any available media that might be accessed by control server 102, and includes volatile and nonvolatile media, as well as, removable and nonremovable media. Computer-readable media might include computer storage media. Computer storage media includes volatile and nonvolatile media, as well as removable and nonremovable media implemented in any method or technology for storage of information, such as computer-readable instructions, data structures, program modules, or other data. In this regard, computer storage media might comprise RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Computer storage media does not comprise signals per se. Combinations of any of the above also may be included within the scope of computer-readable media.

The computer storage media discussed above and illustrated in FIG. 1, including database cluster 104, provide storage of computer-readable instructions, data structures, program modules, and other data for the control server 102. In some embodiments, data cluster 104 takes the form of a cloud-based data store, and in some embodiments is accessible by a cloud-based computing platform.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and providers' offices. Providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like.

The remote computers 108 might also be physically located in nontraditional medical care environments so that the entire healthcare community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

In some embodiments, remote computers 108 comprise computing-devices that are part of a cloud-computing platform. For example, the control server 102 might operate in a computer network 106 hosted as part of a cloud service (e.g., AMAZON WEB SERVICES, GOOGLE HOSTING, IBM BLUEMIX). In some embodiments, a remote computer 108 is associated with a health records data source such as an electronic health record (EHR) system of a hospital or medical organization, a health information exchange EHR, insurance provider EHR, ambulatory clinic EHR, or patient-sensor, or other data source, and facilitates accessing data of the source and communicating the data to control server 102 and/or other computing devices on a cloud computing platform, including other remote computers 108.

Exemplary computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networked environment, program modules or portions thereof might be stored in association with the control server 102, the database cluster 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

In some embodiments, control server 102 is a computing system or platform made up of one or more computing devices. Embodiments of control server 102 may be a distributed computing system, a centralized computing system, a single computer such as a desktop or laptop computer or a networked computing system. Thus, in some embodiments, control server 102 comprises a multi-agent computer system with software agents.

Turning now to FIGS. 2A-2C, an exemplary framework of an auto-adjudication system 200 is shown, in accordance with an aspect of the present invention. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory. The auto-adjudication system 200 may be implemented via any type of computing device, such as computing device 100 described above with reference to FIG. 1, for example.

The auto-adjudication system 200 generally operates to provide frictionless processing of encounters that provides real-time financial transaction and reduces intermediary costs associated with previous systems. In other words, the auto-adjudication system 200 enables specific encounters to bypass normal, standard encounter financial processing checks to ensure clean claims. For example, auto-adjudication system 200 enables specific encounters to bypass insurance verification billing, code validation, and/or claim scrubbing. Additionally, the auto-adjudication system 200 enables the encounter to be automatically adjudicated with an EFT into the bank account of the provider. Accordingly, the provider can be reimbursed in real-time and overall costs are reduced.

As shown in FIGS. 2A-2C, the auto-adjudication system 200 includes, among other components not shown, the patient accounting component 202, the analytics component 204, the financial hub 222, or the payer/partner system 268. It should be understood that the auto-adjudication system 200 shown in FIG. 2 is an example of one suitable computing system architecture. Each of the components shown in FIG. 2 may be implemented via any type of computing device, such as computing device 100 described with reference to FIG. 1, for example.

The components may communicate with each other via a network, which may include, without limitation, one or more local area networks (LANs) and/or wide area networks (WANs). Such networking environments are commonplace in offices, enterprise-wide computer networks, intranets, and the Internet. It should be understood that any number of patient accounting components, analytics components, financial hubs, or payer/partner systems may be employed within the auto-adjudication system 200 within the scope of the present disclosure. Each may comprise a single device or multiple devices cooperating in a distributed environment. For instance, the patient accounting component 202, the analytics component 204, the financial hub 222, or the payer/partner system 268 may be provided via multiple devices arranged in a distributed environment that collectively provide the functionality described herein. In other embodiments, a single device may provide the functionality of multiple components of the auto-adjudication system 200. For example, a single device may provide the patient accounting component 202 and the analytics component 204. Additionally, other components not shown may also be included within the network environment.

Generally, with reference to FIG. 2A, the patient accounting component 202 enables the encounter to be created. As described herein, the encounter corresponds to a billable service provided to a patient. The billable service includes a program bundle 206. The program bundle 206 includes a set of conditions that, if satisfied, enable a billable service to be auto-adjudicated. Additionally the program bundle may include real-time decision support integrated in clinical workflows that ensures adherence to evidence-based standards. The set of conditions may include a single rule set available for all participants (e.g., supplied by rules engine adaptor 216) that enables coding for billing, insurance verification, claim scrubbing, and/or self-pay collection to be bypassed. For example, if a patient is being provided an annual examination, the conditions may require the patient not to have had an annual exam within the last twelve months. In another example, if a patient is being provided a colonoscopy, the conditions may require the patient to be a certain age and not to have had a colonoscopy within a certain time period. Other conditions may require the patient be receiving care in a narrow network or IDN. If each of these conditions are satisfied, the encounter may be automatically adjudicated and coding for billing, insurance verification, claim scrubbing, and/or self-pay collection may be bypassed.

The financial hub 222 generally operates to receive the program data (i.e., the encounter and the program bundle). In this way, the program data is published (via the enterprise service bus 214) for the financial hub 222. As part of this process, the data may be normalized and prepared (e.g., by data transformation component 212) for evaluation by the financial hub 222. Although multiple program bundles 226, 228 are illustrated, they represent the same program bundle communicated asynchronously to each of payer profile component 232, rules engine payer rules component 236, and auto-adjudication service 240. Similarly, multiple enterprise service buses 212, 218 are illustrated that, while representing a single enterprise service bus, differentiate between data flow communicated from the originating system to the financial hub and data flow communicated from the financial hub to the originating system.

As shown in FIG. 2B, the program data 226 is initially routed (e.g., by transaction routing component 224) to payer profiles component 232 and rules engine payer rules 236. Payer profiles component 232 confirms whether the payer participates in auto-adjudication. To do so, the payer profiles component 232 may compare the payer to a payer profile database 234. The rules engine payer rules 236 evaluates the rule set for auto-adjudication criteria that may be maintained in auto-adjudication criteria database 238.

The program data 228 is also routed to auto-adjudication service 240. Initially, the contract management adaptor 242 is queried for a contract price for the codes (corresponding to the billable service) and a health plan of the patient. At this point, remittance data is created by program bundle remittance data component 248 for a pre-funded sweep account 270 of the payer/partner system 268 and the process of electronic funds transfer can be initiated. Additionally, a post adjudication data notice 264 is created by program bundle data post adjudication component 246 for the payer/partner system 268. With reference to FIG. 2C, rules engine adaptor 266 may facilitate communicating the remittance data and the post adjudication data notice 264 to the payer/partner system 268.

A billing record and a de-normalized data stream for analytics is created by billable event tracking component 250 and operational analytics data streaming component 260, respectively. Each of the billing record and the de-normalized data stream for analytics are include in the program data 230 that is published for the originating system via enterprise service bus 218. The data stream for analytics may be de-normalized by data transformation component 220. The billing record 208 is communicated to the originating system (e.g., the patient accounting component 202) and the data stream for analytics 210 is communicated to the analytics component 204.

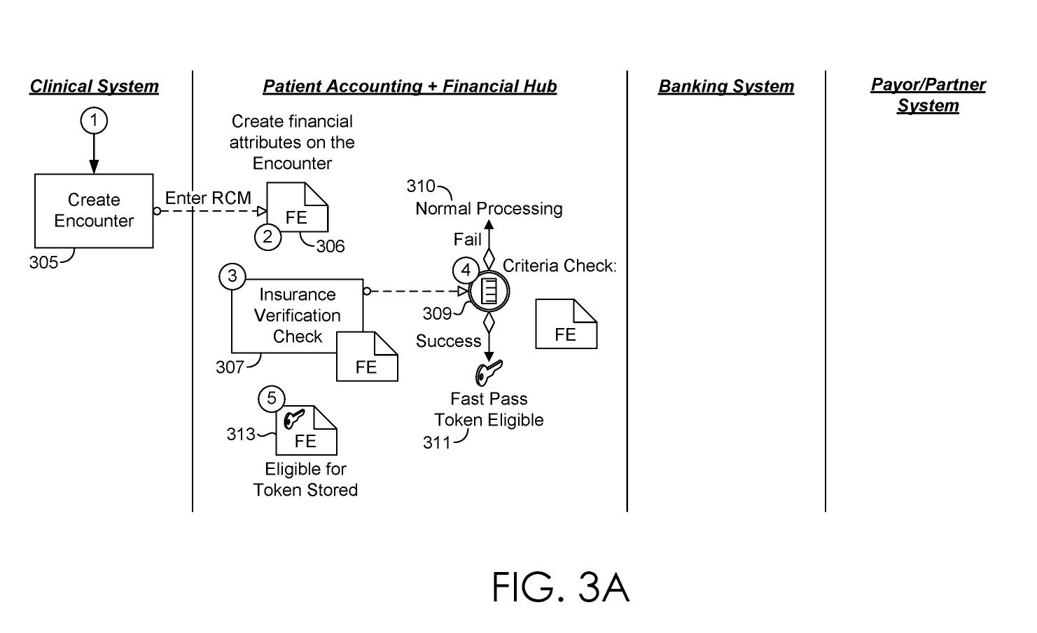

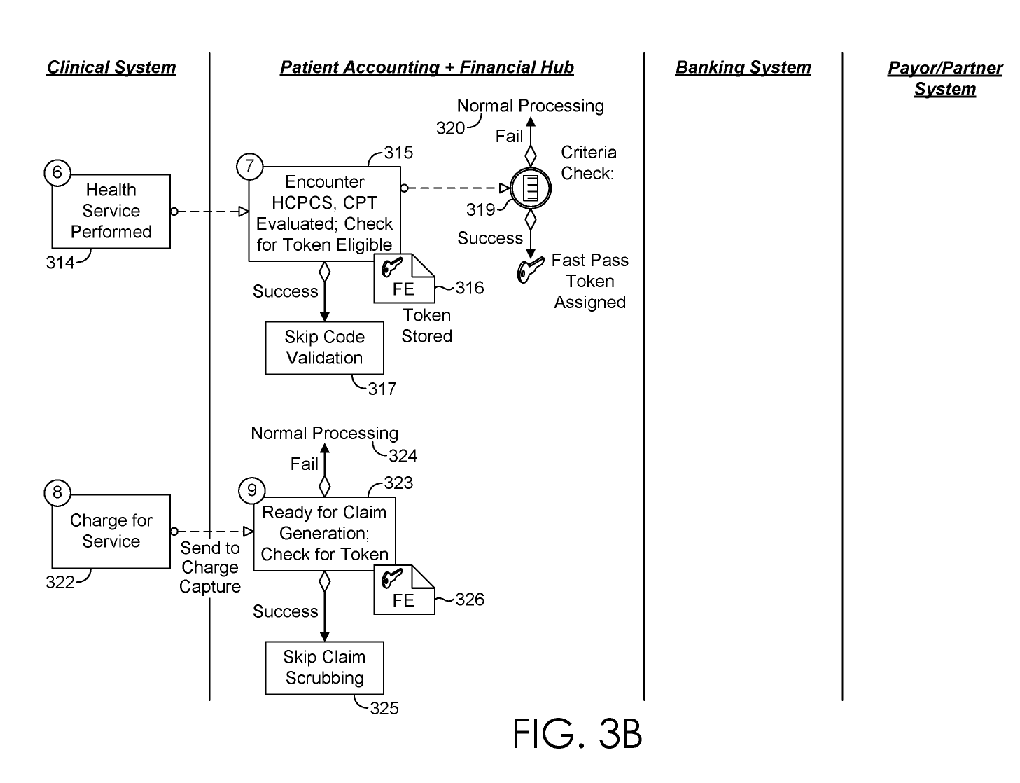

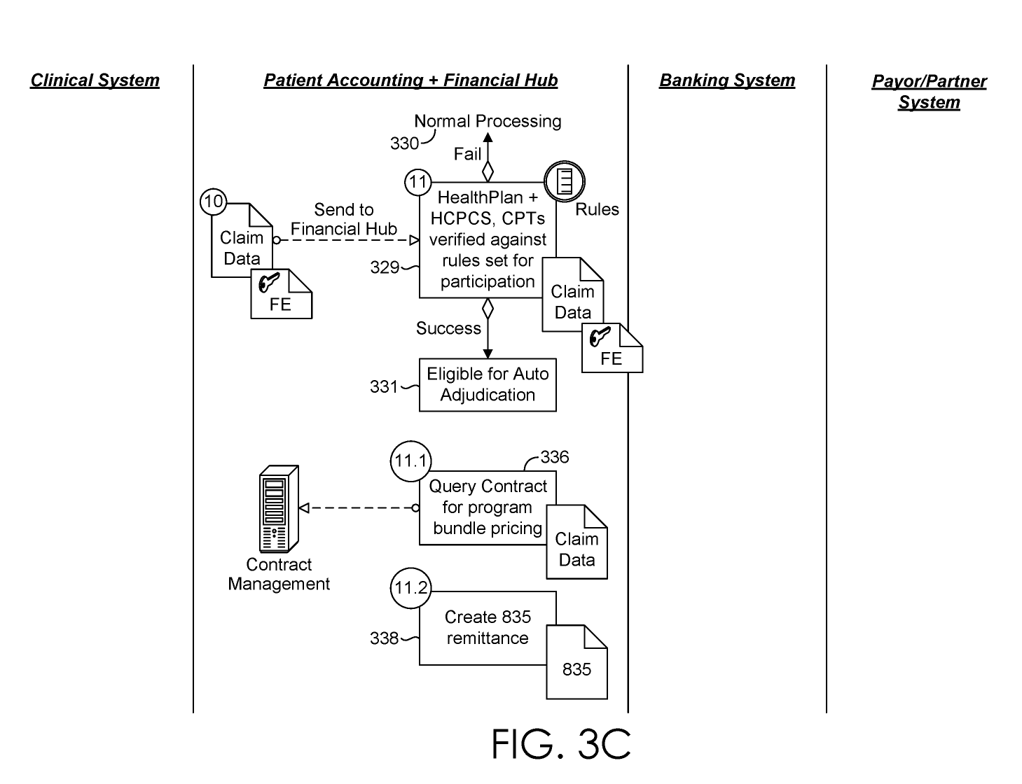

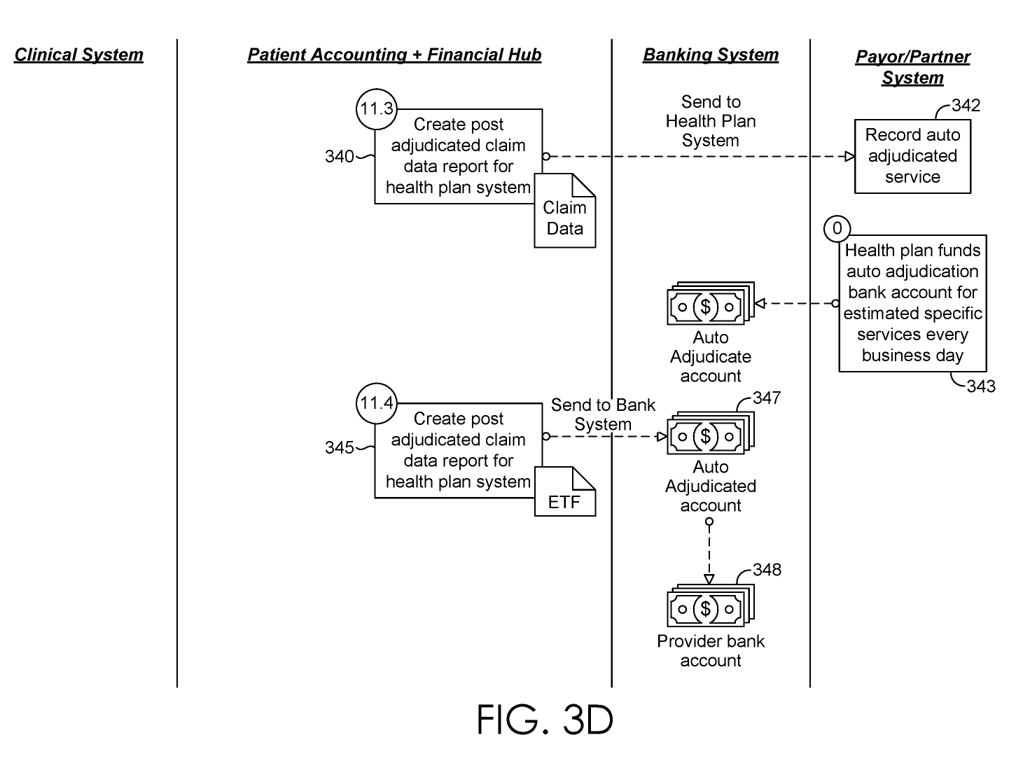

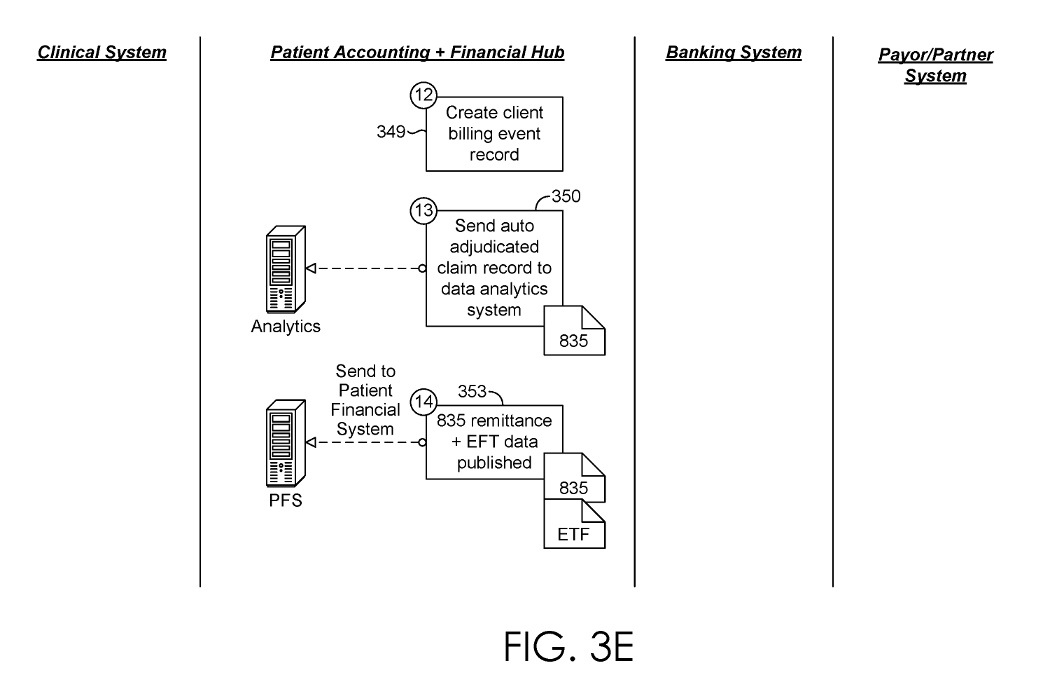

Referring now to FIGS. 3A-3E, an exemplary flow diagram is provided illustrating a method for auto-adjudication, in accordance with an embodiment of the present invention. The method may be performed by any computing device (such as computing device described with respect to FIG. 1) with access to an auto-adjudication system (such as the one described with respect to FIGS. 2A-2C) or by one or more components of the auto-adjudication system.

Initially, with reference to FIG. 3A, an encounter is created at a clinical system at step 305 and communicated to revenue cycle management system such as patient accounting component of FIG. 2. Financial attributes are created for the encounter at step 306. Prior to the insurance verification check, at step 307, it is determined if the encounter is fast pass token eligible at step 309. If the encounter is not fast pass token eligible, normal insurance verification of the encounter proceeds, at step 310. If the encounter is fast pass token eligible, as shown at step 311, the fast pass token is stored with the encounter at step 313 and insurance verification is performed.

Referring to FIG. 3B, after the billable service for the encounter has been performed, as shown at step 314 (which may be communicated by clinical system), the encounter is communicated back to the revenue cycle management system for code validation, at step 315. Initially, the encounter is evaluated for fast pass token eligibility at step 319. If the encounter is not fast pass token eligible, normal code validation of the encounter proceeds, at step 320. If the encounter is fast pass token eligible, the fast pass token is assigned and stored with the encounter at step 316 and code validation is bypassed at step 317.

At this point, the clinical system is ready to charge for the service, as shown at step 322. This is communicated to the revenue cycle management system for charge capture, where the claim is generated. Again, the encounter is evaluated for existence of a fast pass token at step 323. If the encounter is not fast pass token eligible, normal claim scrubbing for the claim proceeds, at step 324. If the encounter has a fast pass token, claim scrubbing is bypassed at step 325.

Next, as illustrated in FIG. 3C, the claim data is communicated to the financial hub. A health plan of the patient and codes are verified against rules set for participation in auto-adjudication, at step 329. If the health plan of the patient and codes indicate the encounter is not eligible for participation in auto-adjudication, normal processing of the claim proceeds, at step 330. If the health plan of the patient and codes indicate the encounter is eligible for participation in auto-adjudication, auto-adjudication of the claim proceeds, at step 331.

A contract management system is queried at step 336 for program bundle pricing. At step, 338 an 835 remittance is created. As shown in FIG. 3D, a post adjudicated claim data report with claim data is created for the payer/partner system at step 340. At step, 342, the post adjudicated claim data report is communicated to the payer/partner system. The payer/partner system estimates specific services each day and funds an auto-adjudication account at step 343.

Next, a post adjudicated claim data report with ETF data is created at step 345. At this point, funds from the auto-adjudicated account are withdrawn, at step 347, and deposited, at step 348, into the provider or health system bank account. Referring now to FIG. 3E, a client billing event record is created at step 349. Additionally, as shown at step 350, an auto-adjudicated claim record is communicated to the data analytics system. Finally, 835 remittance data and EFT data is published, at step 353, to the patient financial system.

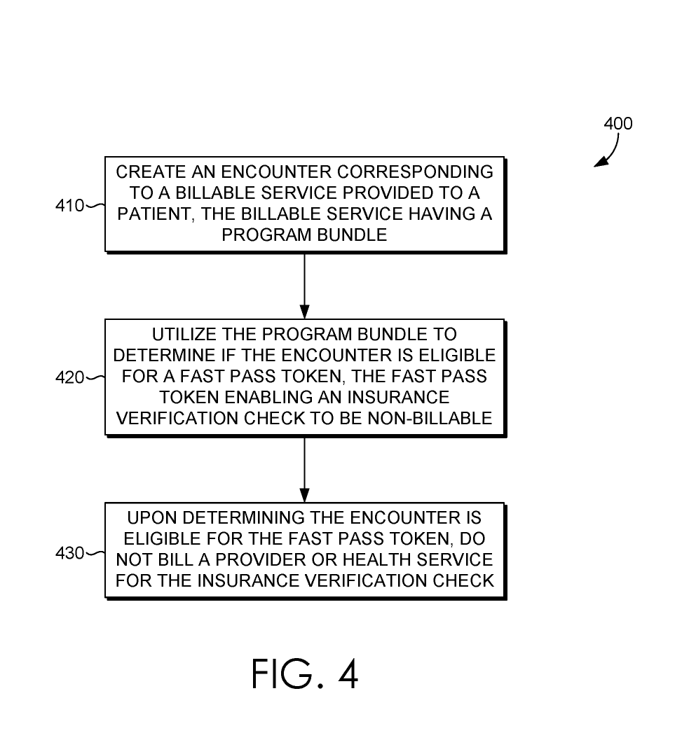

Turning now to FIG. 4, a flow diagram is provided illustrating a method 400 for for bypassing insurance verification, in accordance with embodiments of the present invention. Method 400 may be performed by any computing device (such as computing device described with respect to FIG. 1) with access to an auto-adjudication system (such as the one described with respect to FIGS. 2A-2C) or by one or more components of the auto-adjudication system.

Initially, at step 410, an encounter corresponding to a billable service provided to a patient is created. The billable service includes a program bundle.

At step 420, the program bundle is utilized to determine if the encounter is eligible for a fast pass token. As described herein, the fast pass token prevents billing a provider or health system for an insurance verification check for the encounter.

In some embodiments, as shown at step 430, upon determining the encounter is eligible for the fast pass token, a payer is not billed for the insurance verification check. A token eligible bit flag may be set for and stored with the encounter. Upon determining the encounter is not eligible for the fast pass token, normal insurance verification check processing may continue.

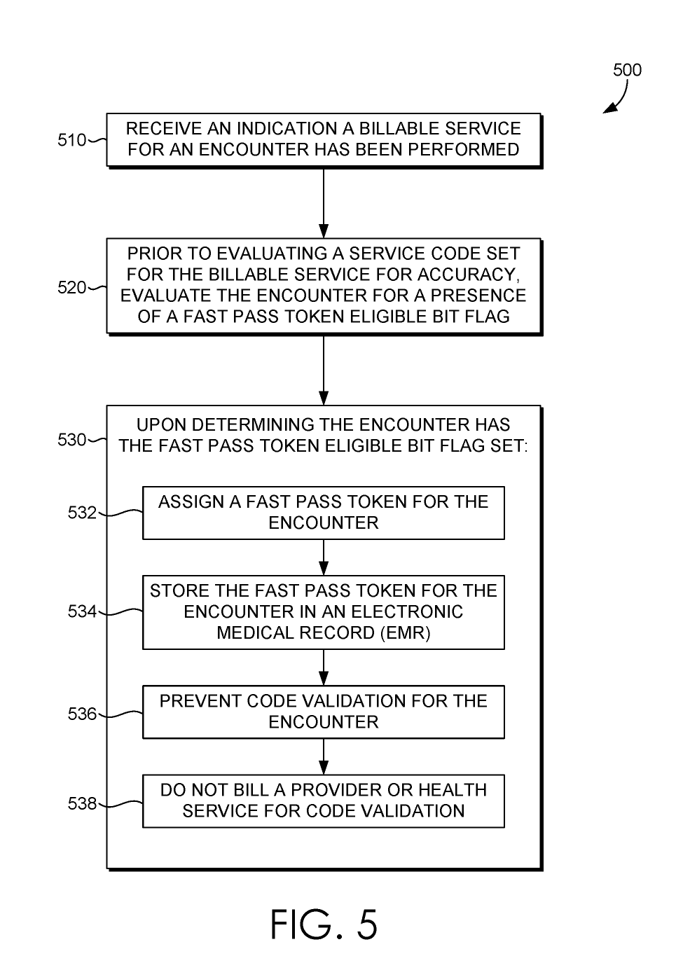

In FIG. 5, a flow diagram is provided illustrating a method 500 for bypassing code validation, in accordance with embodiments of the present invention. Method 500 may be performed by any computing device (such as computing device described with respect to FIG. 1) with access to an auto-adjudication system (such as the one described with respect to FIGS. 2A-2C) or by one or more components of the auto-adjudication system.

Initially, at step 510, an indication a billable service for an encounter has been performed is received.

At step 520, prior to evaluating a code set for the billable service for accuracy, the encounter is evaluated for a presence of a fast pass token eligible bit flag.

At step 530, upon determining the encounter has the fast pass token eligible bit flag set: determine a code set for a billable service is associated with a list of services eligible for a fast pass token and a fast pass token for the encounter is assigned, at step 532; the fast pass token for the encounter is stored, at step 534, in an electronic medical record (EMR); code validation for the encounter is prevented at step 536; and a payer is not billed for code validation at step 538.

In some embodiments, the code set is one of a Healthcare Common Procedure Coding System (HCPCS) code set or a Current Procedural Terminology (CPT) code set. A program bundle corresponding to the encounter may be utilized to determine if the encounter is eligible for the fast pass token. If it is eligible, the token eligible bit flag may be set for and stored with the encounter. Upon determining the encounter does not have the fast pass token eligible bit flag set, normal code validation may continue.

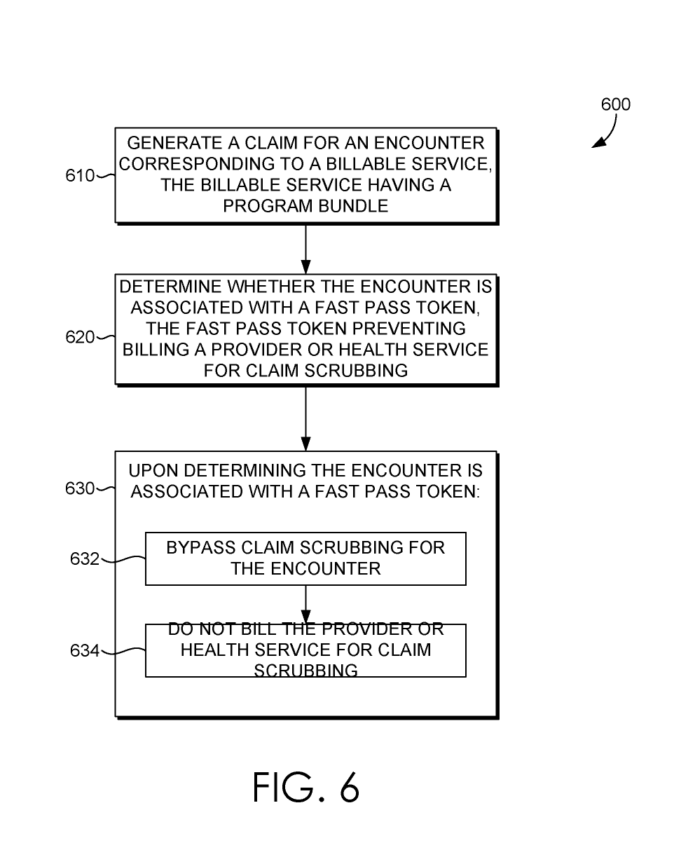

Referring now to FIG. 6, a flow diagram is provided illustrating a method 600 for bypassing claim scrubbing, in accordance with embodiments of the present invention. Method 600 may be performed by any computing device (such as computing device described with respect to FIG. 1) with access to an auto-adjudication system (such as the one described with respect to FIGS. 2A-2C) or by one or more components of the auto-adjudication system.

Initially, at step at step 620, it is determined whether an encounter is associated with a fast pass token. The fast pass token prevents billing a payer for claim scrubbing.

At step 630, upon determining the encounter is associated with a fast pass token: claim scrubbing for the encounter is bypassed at step 632; and the payer is not billed for claim scrubbing at step 634.

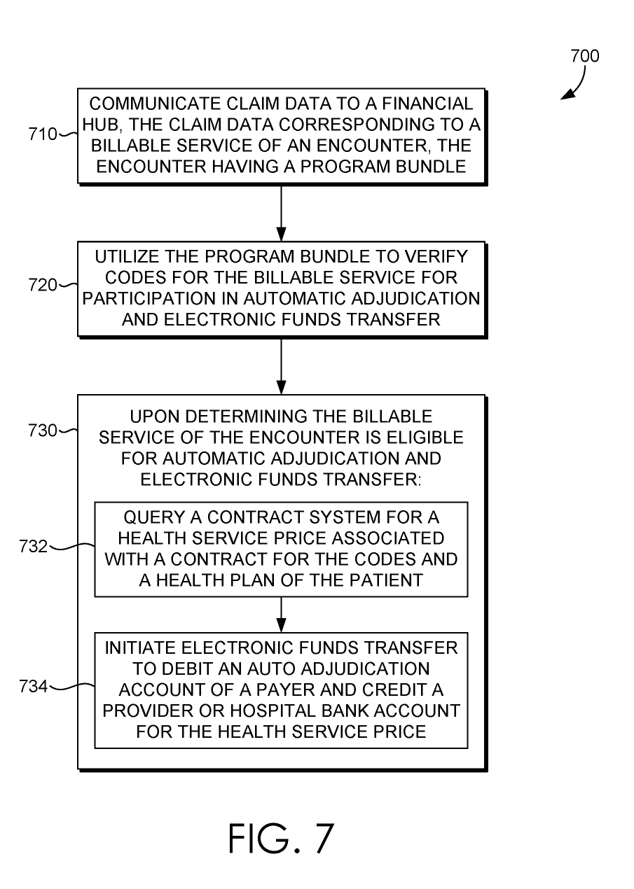

Turning now to FIG. 7, a flow diagram is provided illustrating a method 700 for auto-adjudication of medical encounters, in accordance with embodiments of the present invention. Method 700 may be performed by any computing device (such as computing device described with respect to FIG. 1) with access to an auto-adjudication system (such as the one described with respect to FIGS. 2A-2C) or by one or more components of the auto-adjudication system.

Initially, at step 710, claim data is communicated to a financial hub. The claim data corresponds to a billable service of an encounter. The encounter includes a program bundle.

At step 720, the program bundle is utilized to verify codes for the billable service for participation in auto-adjudication and electronic funds transfer.

At step 730, upon determining the billable service of the encounter is eligible for auto-adjudication and electronic funds transfer: a contract management system is queried, at step 732, for a health service price associated with a contract for the codes and a health plan of a patient; and electronic funds transfer is initiated, at step 734, to debit an auto-adjudication account of a payer and credit a provider or hospital bank account for the health service price.

In some embodiments, a billing record is created for an auto-adjudicated transaction fee. The billing record includes 835 remittance data and electronic funds transfer data. The billing record is communicated to an analytics system and a patient financial system. Additionally, a post adjudicated claims data report may be created for a payer system. A notice, including the post adjudicated claims data report may be communicated to a health plan system of the health plan.

In some embodiments, upon determining the medical service for encounter is not eligible for auto-adjudication and electronic funds transfer, continuing with normal claim processing by communicating the encounter to a healthcare clearinghouse or to the health plan of the patient.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. A system for utilizing frictionless processing auto-adjudication of medical encounters, the system comprising:
a processor; and
a computer storage medium storing computer-usable instructions that, when used by the processor, cause the processor to:
communicate claim data to a financial hub, the claim data corresponding to a billable service of an encounter, the encounter having a program bundle;
utilize the program bundle to verify codes for the billable service for participation in auto-adjudication and electronic funds transfer;
upon determining the billable service of the encounter is eligible for auto-adjudication and electronic funds transfer:
(a) query a contract management system for a health service price associated with a contract for the codes and a health plan of a patient; and
(b) initiate electronic funds transfer to debit an auto-adjudication account of a payer and credit a provider or hospital bank account for the health service price.

2. The system of claim 1, wherein the codes are Healthcare Common Procedure Coding System (HCPCS) or Current Procedural Terminology (CPT).

3. The system of claim 1, further comprising initiating processing to charge for the billable service associated with the encounter.

4. The system of claim 3, further comprising creating a post adjudicated claims data report for a payer system.

5. The system of claim 4, further comprising communicating a notice to a health plan system of the health plan.

6. The system of claim 5, further comprising creating a billing record for auto-adjudicated transaction fee, the billing record having 835 remittance data and electronic funds transfer data

7. The system of claim 6, further comprising communicating the billing record to an analytics system and a patient financial system.

8. The system of claim 5, further comprising, upon determining the medical service for encounter is not eligible for auto-adjudication and electronic funds transfer, continuing with normal claim processing by communicating the encounter to a healthcare clearinghouse or to the health plan of the patient.

9. The system of claim 1, further comprising, upon determining the encounter is eligible for the fast pass token, not billing a provider or health service for the insurance verification check.

10. The system of claim 9, further comprising setting a token eligible bit flag for this encounter.

11. The system of claim 10, further comprising storing the token eligible bit flag.

12. The system of claim 11, receiving an indication the billable service has been performed.

13. The system of claim 12, further comprising, prior to evaluating a code set for accuracy, evaluating the encounter for the existence of the fast pass token eligible bit flag.

14. The system of claim 13, further comprising, upon determining the encounter has the fast pass token eligible bit flag set:
assigning a fast pass token for the encounter; and
storing the pass token for the encounter in an electronic medical record.

15. The system of claim 14, further comprising:
preventing HCPCS, CPT code validation for the encounter; and
not billing the payer for code validation.

16. The system of claim 15, further comprising, during claim generation, determine whether the encounter is associated with a fast pass token, the fast pass token preventing billing a payer for claim scrubbing.

17. The system of claim 16, further comprising, upon determining the encounter is associated with a fast pass token bypassing claim scrubbing for the encounter.

18. The system of claim 16, further comprising not billing the payer for claim scrubbing.

19. A computerized method for utilizing frictionless processing for auto-adjudication of medical encounters, the method comprising:
communicating claim data to a financial hub, the claim data corresponding to a billable service of an encounter, the encounter having a program bundle;
utilizing the program bundle to verify codes for the billable service for participation in auto-adjudication and electronic funds transfer;
upon determining the billable service of the encounter is eligible for auto-adjudication and electronic funds transfer:
(a) querying a contract management system for a health service price associated with a contract for the codes and a health plan of a patient; and
(b) initiating electronic funds transfer to debit an auto-adjudication account of a payer and credit a provider or hospital bank account for the health service price.

20. One or more computer storage media having computer-executable instructions embodied thereon that, when executed by a computer, causes the computer to perform operations to utilize frictionless processing for auto-adjudication of medical encounters, the operations comprising:
communicating claim data to a financial hub, the claim data corresponding to a billable service of an encounter, the encounter having a program bundle;
utilizing the program bundle to verify codes for the billable service for participation in auto-adjudication and electronic funds transfer;
upon determining the billable service of the encounter is eligible for auto-adjudication and electronic funds transfer:
(a) querying a contract management system for a health service price associated with a contract for the codes and a health plan of a patient; and
(b) initiating electronic funds transfer to debit an auto-adjudication account of a payer and credit a provider or hospital bank account for the health service price.
